(19) United States
(12) Patent Application Publication
(10) Pub. No.: US 2010/0150920 A1
(43) Pub. Date: Jun. 17, 2010

(54) System and Method for Identifying Biomarkers in Ocular Fluid That Are Indicative of Ocular Disease

(76) Inventor: Bert Glaser, Towson, MD (US)

Correspondence Address: LAMORTE & ASSOCIATES P.C., P.O. BOX 434, YARDLEY, PA, 19067, US

(21) Appl. No.: 12/631,800

(22) Filed: Dec. 4, 2009

Related U.S. Application Data
(60) Provisional application No. 61/120,014, filed on Dec. 4, 2008.
(60) Provisional application No. 61/120,152, filed on Dec. 5, 2008.

(51) Int. Cl. A61K 39/395; C40B 30/04

(52) U.S. Cl. 424/133.1; 506/9

(57) ABSTRACT

A system and method for testing ocular fluid, such as vitreous fluid. The testing can be used to both determine if an ocular disease is present and to quantify the effectiveness of a treatment for that ocular disease. A sample of ocular fluid is drawn from a patient and tested for the presence of targeted biomarkers and for the level of those biomarkers. The level of the biomarkers in the patients sample is compared to the normal range. If the level of the targeted biomarker falls outside the normal range, an abnormal condition is recognized. The effectiveness of any treatment can be analyzed by repeating the testing process. After a treatment, ocular fluid is again drawn. The biomarker levels can be identified and compared to those taken before treatment began. If the level of the targeted biomarkers trends toward normal, the treatment is known to be effective.

### RELATED APPLICATIONS

This application claims the benefit of Provisional Patent Application No. 61/120,014, entitled Biomarkers In Vitreous Fluid Of Patients With Ocular Disease, filed Dec. 4, 2008.

This application also claims the benefit of Provisional Patent Application No. 61/120,152, entitled, Biomarkers In Vitreous Fluid Of Patients With Ocular Disease, filed Dec. 5, 2008.

### BACKGROUND OF THE INVENTION

1. Field of the Invention

In general, the present invention relates to systems and methods that test bodily fluids for biomarker indicators that identify an underlying disease or abnormal condition. More particularly, the present invention relates to systems and methods that test fluids of the eye in order to identify potential abnormal conditions of the eye.

2. Prior Art Description

There are many diseases of the eye that can cause lost vision. Ocular diseases include Age-related Macular Degeneration, Age-related macular degeneration, idiopathic choroidal neovascularization, epiretinal membrane, macular hole, retinal detachment, vitreous hemorrhage, diabetic retinopathy, epiretinal membrane, retinal angiomatous proliferation, proliferative diabetic retinopathy, choroidal neovascularization, glaucoma, cataract, and uveitis.

Age-related macular degeneration (AMD) is one of the leading causes of vision loss and blindness in individuals over age 60 in the developed world. The manifestations of AMD are classified into two forms: non-exudative (dry) and exudative (wet or neovascular), which is characterized by choroidal neovascularization (CNV). In wet AMD, the choroidal capillaries proliferate and penetrate Bruch's membrane to reach the retinal pigment epithelium (RPE). Capillary leakage, fovea swelling, fibrous metaplasia and scarring may lead to permanent vision loss.

Idiopathic choroidal neovascularization (CNV) is an eye disease wherein new blood vessels that originate from the choroid grow through a break in the Bruch membrane into the sub-retinal pigment epithelium (sub-RPE) or subretinal space. Choroidal neovascularization (CNV) is a major cause of visual loss. Virtually any pathologic process that involves the RPE and damages the Bruch membrane can be complicated by CNV. Recently, a protein derived from the RPE, pigment epithelium derived factor (PEDF), was found to have an inhibitory effect on ocular neovascularization. Another peptide, vascular endothelium growth factor (VEGF), is a well-known ocular angiogenic factor that may play a role. The symptoms of CNV include painless loss of vision, metamorphopsia, paracentral or central scotoma, apparent change in image size, subretinal blood, subretinal fluid, lipid exudation, retinal pigment epithelial detachment and subretinal fibrosis (disciform scar).

Diabetic retinopathy is the most common diabetic eye disease and a leading cause of blindness in American adults. Blindness is caused by changes in the blood vessels of the retina. Epiretinal membrane (also referred to as preretinal membrane, cellophane maculopathy, retina wrinkle, surface wrinkling retinopathy, premacular fibrosis, and internal limiting membrane disease) is scar tissue that has formed on the eye's macula, located in the center of the eye's light-sensitive tissue called the retina. Macular hole (also known as macular cyst, retinal hole, retinal tear, and retinal perforation) is a small break in the macula, located in the center of the eye's light-sensitive tissue called the retina. Although some macular holes can seal themselves and require no treatment, surgery is necessary in many cases to help improve vision.

Treatments for ocular diseases are rapidly evolving and some now target specific biochemical events such as angiogenesis. The use of pharmacotherapy targeting vascular endothelial growth factor (VEGF) has been shown to slow vision loss and even lead to vision improvement in some patients with wet macular degeneration. Currently the three approved anti-VEGF therapies are pegaptanib (Macugen®), ranibizumab (Lucentis®), and bevacizumab (Avastin®). Ranibizumab and bevacizumab (a full-length recombinant, humanized, monoclonal antibody) bind and inhibit all forms of VEGF-A.

Current experience with inhibitors of VEGF reveals that although these therapies are successful in many patients, the response to anti-VEGF therapy varies greatly and it is unknown what biological parameters play a role in determining response. Despite the increasingly sophisticated imaging technology and treatment modalities available, there is a need for technologies that can guide initiation of treatment at the earliest possible time, preferably prior to the loss of vision. A need also exists for a system and method that can predict and guide the need for re-treatment before a recurrence. Lastly, a need exists for a system and method that help physicians understand other factors that are involved with patients that have suboptimal response to treatment or are recalcitrant to treatment with the goal of developing new targets for drug development. These needs are met by the present invention as described below.

### SUMMARY OF THE INVENTION

The present invention is a system and method for testing ocular fluid, such as vitreous fluid. The testing can be used to both determine if an ocular disease is present and to quantify the effectiveness of a treatment for that ocular disease.

In accordance with the present invention, a sample of ocular fluid is drawn from a patient expected to have an ocular disease. The ocular fluid sample is tested for the presence of targeted biomarkers and for the level of those biomarkers in the sample. The biomarkers can be apoptosis factors, growth factors, angiogenesis factors, and inflammation factors. The preferred biomarkers are selected from a group consisting of C-abl, CC9 D330, VEGFR Y1175, VEGFR Y996, PEDF, TNF-alpha, VEGFA, VEGFR Y951, IL-1B, and combinations thereof.

Normal ranges for targeted biomarkers are obtained from at least one health subject. If the range is taken from a small sampling, it is preferred that the sample share a certain common demographics with the patient. The level of the biomarkers in the patients sample is compared to the normal range. If the level of the targeted biomarker falls outside the normal range, an abnormal condition is recognized that may be indicative of disease.

If disease is diagnosed, the patient can be treated in numerous ways. The effectiveness of any treatment can be analyzed by repeating the testing process. After a treatment has had time to cause effect, ocular fluid can be drawn from the patient. The biomarker levels can be identified and compared to those taken before treatment began. If the level of the targeted biomarkers trends toward normal, the treatment is known to be effective. If the level of targeted biomarkers does not change or trends away from normal, the treatment is known to be ineffective.

### BRIEF DESCRIPTION OF THE DRAWINGS

For a better understanding of the present invention, reference is made to the following description of an exemplary embodiment thereof, considered in conjunction with the accompanying drawings, in which:

FIG. 1 is bock diagram schematic of an exemplary embodiment of the present invention;

FIG. 2A is a histogram for an example responsive patient that plots protein endpoint verses relative mean intensity (RMI);

FIG. 2B is a histogram for an example non-responsive patient that plots protein endpoint verses relative mean intensity (RMI);

FIG. 3 is part one of a table that lists data regarding biomarker levels in different patients and changes to those biomarker levels in response to therapeutic agents;

FIG. 4 is part two of a table that lists data regarding biomarker levels in different patients and changes to those biomarker levels in response to therapeutic agents; and

FIG. 5 is part three of a table that lists data regarding biomarker levels in different patients and changes to those biomarker levels in response to therapeutic agents.

### DETAILED DESCRIPTION OF THE INVENTION

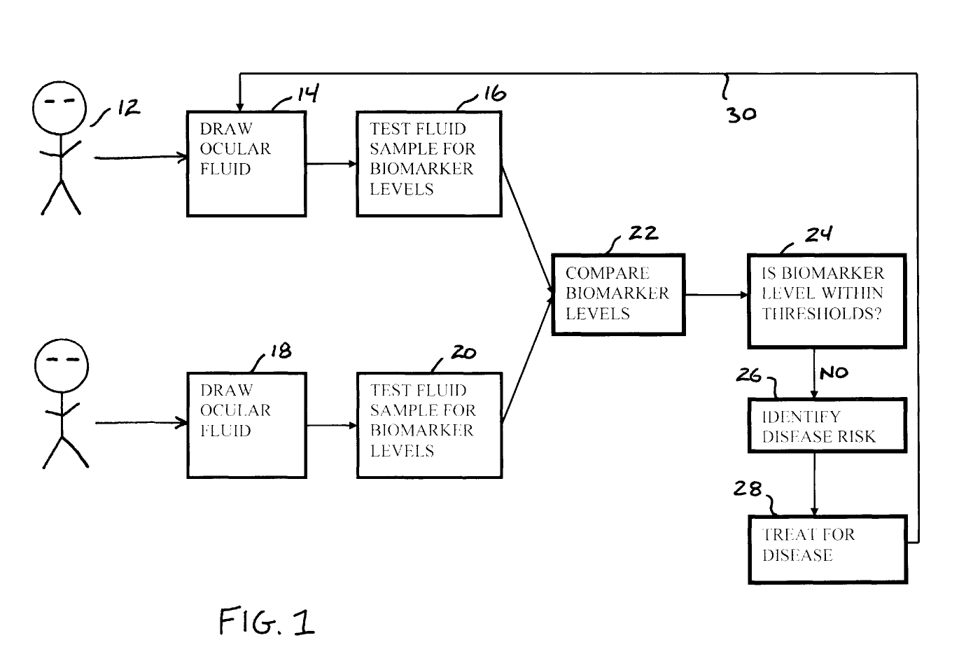

Referring to FIG. 1, an overview of the present invention system and method is shown. The purpose of the system and method is to determine if a particular patient 12 has an ocular disease and/or if the patient is responding to treatment for an ocular disease. A variety of ocular related diseases are encompassed. These include without limitation Dry Age-related Macular Degeneration, Wet Age-related macular degeneration (AMD), idiopathic choroidal neovascularization (CNV), epiretinal membrane, macular hole, retinal detachment, vitreous hemorrhage, diabetic retinopathy, epiretinal membrane, retinal angiomatous proliferation, proliferative diabetic retinopathy, choroidal neovascularization, glaucoma, cataract, and uveitis.

The diagnosis of an ocular disease is a multi-step process. As is indicated by Block 14, a volume of ocular fluid is drawn from a patient's eye. Any ocular fluid can be analyzed in accordance with the present invention, including, vitreous fluids; aqueous fluids; retinal blood, such as blood present in the choroid; and tears, including tears extracted from the lacrimal sac. Fluids can be extracted by surgical vitrectomy procedures or by fine needle aspiration of the fluid. In a preferred embodiment, vitreous fluid of the eye is obtained by aspiration. Preferably, no aqueous fluid from the anterior chamber is used. The amount of a fluid sample collected can be between 5 to 200 microliters. The ocular fluid collected is preferably free of cells. Fluid samples may be rendered free of cells by means known in the art including centrifugation and removal of the cell free supernatant. Using processes that are later described, the ocular fluid sample is then tested to determine the level of various biomarkers in that fluid. See Block 16.

Knowing the level of a targeted biomarker in a sample is not enough. The level of targeted biomarker is relativistic. Biomarkers levels naturally vary through different demographics. For example, the levels of different biomarkers differ in different age groups, gender groups and ethnicity groups.

To quantify the level of biomarkers in the vitreous fluid of a particular patient, those levels are compared to normal ranges for those biomarkers. The normal ranges may be determined statistically from a large sampling of healthy individuals or generally the same age, gender and ethnicity. Alternatively, the normal ranges may be obtained by testing a close relative to the patient, preferably a same gender sibling. In either scenario, the methodology outlined in FIG. 1 is used. Vitreous fluid is drawn from a healthy subject, or subjects. See Block 18. The healthy vitreous fluid is tested to determine the level of various targeted biomarkers. See Block 20. The levels of biomarkers in the healthy sample are then set as the normal levels. The level of targeted biomarkers in the patient are then compared to the normal levels. See Block 22. The normal levels are ranges with upper and lower thresholds. If the level of a biomarker from a patient falls above or below the threshold of the normal range, then that biomarker level can be considered “abnormal”. See Block 24.

With an abnormal biomarker level identified, a diagnosis of a particular ocular disease can be made. See Block 26. The patient can then be treated for that ocular disease while the disease is still in its early stages. See Block 28. The treatment of the disease may be surgical. However, the administration of a therapeutic agent is most often the first course of action.

If a therapeutic agent is used to treat the ocular disease, then the present invention system can be cycled to determine the effective or ineffectiveness of the therapeutic agent that has been administered. As is indicated by arrow 30 in FIG. 1, one a patient has received treatment for an ocular disease, the ocular fluid can again be collected and tested. If the level of a previously abnormal biomarker falls back into a normal range, it is known that the treatment has been effective. Conversely, if the level of the abnormal biomarker does not change or becomes worse, then it is know that the course of treatment is ineffective.

In addition to changes in the level of targeted biomarkers, improvements and setbacks can be ascertained through various clinical responses. For example, improvements in visual acuity are a positive clinical response. In another aspect, changes in the height of sub-retinal fluid or a decrease in central retinal thickness may be considered a positive response. Other positive clinical responses include a decrease in the area of leakage on a fluorescein angiography. Likewise, so would be the closure of a retinal angiomatous proliferation (RAP) lesion.

In FIG. 1, the testing of the ocular fluid to find at least one targeted biomarker is shown by Block 16. In the present invention, a biomarker can indicate a change in expression or activity state of a protein that correlates with the risk or progression of a disease, or with the susceptibility of the disease to a given treatment. A variety of biomarkers can identify an ocular disease. For example without limitation, BAD Ser112, Bcl-2, C-abl, CC9 D330, Fadd 5194, FGF-R, PDGFRβ Y716, PDGFRβ Y751, VEGFA, VEGFR Y951, VEGFR Y996, VEGFR Y1175, C-kit Y703, C-kit Y719, PEDF, MMP-2, MMP-9, TNF-alpha, IL-1B and eNOs S1177. The biomarkers may be used singly or as part of a group as proxy indicator of a biologic state or of effectiveness of a treatment. Biomarkers may also be characterized by known molecular relationships, signaling pathways and similarity to homologous proteins. In one aspect, a level of phosphorylated biomarker is determined. In another aspect, the level of a phosphorylated biomarker is compared to the non-phosphorylated form. In yet another aspect, a biomarker may be grouped by involvement in a physiological process such as inflammation factors, angiogenesis factors, apoptosis factors, or may grouped by a biological property such as being a growth factor. Non-limiting examples of biomarkers of the present invention that are implicated in inflammation are TNF-alpha, IL-1B and eNOs S1177. Non-limiting examples of biomarkers of the present invention related to angiogenesis are C-kit Y703, C-kit Y719, PEDF, MMP-2 and MMP-9. Apoptosis related biomarkers include BAD Ser112, Bcl-2, C-abl, CC9 D330 and Fadd 5194. Biomarkers that are known growth factors include FGF-R, PDGFR Y716, PDGFR Y751, VEGFA, VEGFR Y951, VEGFR Y996, and VEGFR Y1175. Various proteins can be considered members of multiple groups, such as VEGF which is both a known growth factor and which is also known to be involved in angiogenesis.

Biomarkers can be distinguished by modifications that make them distinct. For example, the VEGF receptor (VEGFR) can be modified on at least three tyrosine residues Y951, Y996, Y1175. Each modification can be a surrogate indicator of which treatment regime will yield a better clinical outcome or the effectiveness of the therapy. In one aspect, high levels of VEGFR Y1175 and VEGFR Y996 together with C-abl, CC9 D330, PEDF and TNF-alpha are predictive of a positive and rapid clinical response to bevacizumab treatment of wet AMD. In another aspect, elevated levels of at least VEGFR Y1175 combined with non-elevated levels of VEGFR Y996 and non-elevated levels VEGFR Y951 predict a RPE Detachment Response to bevacizumab. In another aspect, elevated levels of VEGFR Y1175, PEDF R1, c-Abl, and TNF-alpha R1 combined with non-elevated levels of VEGFR Y996 and VEGFR Y951 reflect a RPE Detachment Response to bevacizumab.

A biomarker level in an ocular fluid can be measured by any know biomarker detection technology. Such technologies include protein microarray, an immunoassay, a ligand binding assay, electrophoresis, and mass spectroscopy of the sample. Immunologic techniques, antibody diagnostics, radioimmunoassays, spectrometry, mass spectrometry, microarrays, western blotting, gel electrophoresis, and labeled or enzyme amplified diagnostic technologies can be employed. In some embodiments, detecting includes a proteomic fingerprint comprising a vitreous fluid polypeptide or unique fragment thereof. Non protein biomarkers such as nucleic acids, glycans, proteoglycans, carbohydrates and steroids may also be detected.

Additionally, detecting can include methods that distinguish variants of the biomarker. For example, an immunologic technique using antibodies directed to modified forms of the biomarker may be employed. In a preferred aspect, antibodies directed to phosphorylated tyrosine residues can be employed allowing the independent detection of VEGFR Y951, VEGFR Y996, and VEGFR Y1175. In another aspect, antibodies may recognize cleaved or proteolyzed proteins while intact proteins are not detected. Alternatively, antibodies may recognize only biomarker in their native state and fail to bind to biomarkers that are denatured.

In the preferred embodiment of the invention, a Reverse Phase Protein Microarray (RPPM) is employed to measure the amounts of a biomarker. In this aspect, total protein content of the samples is first measured by standard methods (e.g. spectrophotometrically using the Bradford method). The samples are then diluted in and denatured prior to dilution in a microtiter plate. The diluted denatured lysates are printed on glass backed nitrocellulose array slides (FAST Slides Whatman, Florham Park, N.J.) using an arrayer (Aushon 2470 equipped with 350 μm pins, Aushon BioSystems, Burlington, Mass.). Each lysate is printed in a dilution curve representing neat, 1:2, 1:4, 1:8, and 1:16 dilutions. A micro dot-blot format has the advantages of high density and minimal amounts of sample material. A RPPM is similar to other common assay methods such as western blot and ELISA and can employ similar detection methods including colorimetric detection via amplification step involving tagged secondary antibodies and enzyme linked antibodies.

Essentially all modes of affinity binding assays are applicable for use in determining a level of a biomarker in a sample. Such methods are rapid, efficient and sensitive. Moreover, affinity binding methods are simple and can be modified to be performed under a variety of clinical settings and conditions to suit a variety of particular needs. Affinity binding assays that are known and can be used in the methods of the invention include both soluble and solid phase formats. A specific example of a soluble phase affinity binding assay is immunoprecipitation using a biomarker selective antibody or other binding agent. Solid phase formats are advantageous for the methods of the invention since they are rapid and can be performed more easily on multiple different samples simultaneously without losing sensitivity or accuracy. Moreover, solid phase affinity binding assays are further amenable to high throughput screening and automation.

Specific examples of solid phase affinity binding assays include immunohistochemical binding assays, immunoaffinity binding assays such as an ELISA and radioimmune assay (RIA). Other solid phase affinity binding assays are known to those skilled in the art and are applicable to the methods of the invention. Although affinity binding assays are generally formatted for use with an antibody binding molecules that is selective for the biomarker or ligand of interest, essentially any binding agent can be alternatively substituted for the selectively binding antibody. Such binding agents include, for example, macromolecules such as polypeptides, peptides, nucleic acid molecules, lipids and sugars as well as small molecule compounds. Methods are known in the art for identifying such molecules which bind selectively to a particular biomarker or ligand and include, for example, surface display libraries and combinatorial libraries. Thus, for a molecule other than an antibody to be used in an affinity binding assay, all that is necessary is for the binding agent to exhibit selective binding activity for a biomarker.

The various modes of affinity binding assays, such as immunoaffinity binding assays, include, for example, immunohistochemistry methods, solid phase ELISA and RIA as well as modifications thereof. Such modifications thereof include, for example, capture assays and sandwich assays as well as the use of either mode in combination with a competition assay format. Choice of which mode or format of immunoaffinity binding assay to use will depend on the intent of the user. Such methods can be found described in common laboratory manuals such as Harlow and Lane, Using Antibodies: A Laboratory Manual, Cold Spring Harbor Laboratory Press, New York (1999).

Formats employing affinity binding can be used in conjunction with a variety of detection labels and systems known in the art to quantitate amounts of biomarkers in the analyzed sample. Detection systems include the detection of bound biomarker by both direct and indirect means. Direct detection methods include labeling of the biomarker-specifically reactive antibody or binding agent. Indirect detection systems include, for example, the use of labeled secondary antibodies and binding agents.

Secondary antibodies, labels and detection systems are well known in the art and can be obtained commercially or by techniques well known in the art. The detectable labels and systems employed with the biomarker-selective binding agent should not impair binding of the agent to the biomarker. Moreover, multiple antibody and label systems can be employed for detecting the bound biomarker-specifically reactive antibody to enhance the sensitivity of the binding assay if desired.

Detectable labels can be essentially any label that can be quantified or measured by analytical methods. Such labels include, for example, enzymes, radioisotopes, fluorochromes as well as chemi- and bioluminescent compounds. Specific examples of enzyme labels include horseradish peroxidase (HRP), alkaline phosphatase (AP), .beta.-galactosidase, urease and luciferase.

A horseradish-peroxidase detection system can be used, for example, with the chromogenic substrate tetramethylbenzidine (TMB), which yields a soluble product in the presence of hydrogen peroxide that is detectable by measuring absorbance at 450 nm. An alkaline phosphatase detection system can be used with the chromogenic substrate p-nitrophenyl phosphate, for example, which yields a soluble product readily detectable by measuring absorbance at 405 nm. Similarly, a .beta.-galactosidase detection system can be used with the chromogenic substrate o-nitrophenyl-.beta.-D-galactopyranoside (ONPG), which yields a soluble product detectable by measuring absorbance at 410 nm, or a urease detection system can be used with a substrate such as urea-bromocresol purple (Sigma Immunochemicals, St. Louis, Mo.). Luciferin is the substrate compound for luciferase which emits light following ATP-dependent oxidation.

Fluorochrome detection labels are rendered detectable through the emission of light of ultraviolet or visible wavelength after excitation by light or another energy source. DAPI, fluorescein, Hoechst 33258, R-phycocyanin, B-phycoerythrin, R-phycoerythrin, rhodamine, Texas red and lissamine are specific examples of fluorochrome detection labels that can be utilized in the affinity binding formats of the invention.

Flourochromes may also be combined with enzyme linked amplification methods in the invention. For example, the Tyramide Signal Amplification (TSA™) is a method wherein horseradish-peroxidase (linked to secondary antibody) catalyzes the deposition and binding of a labeled (e.g. biotin or fluorescent moieties) tyramide in situ through a free radical based mechanism.

Chemiluminescent as well as bioluminescent detection labels are convenient for sensitive, non-radioactive detection of a biomarker and can be obtained commercially from various sources such as Amersham Lifesciences, Inc. (Arlington Heights, Ill.).

Signals from detectable labels can be analyzed, for example, using a spectrophotometer to detect color from a chromogenic substrate; a fluorometer to detect fluorescence in the presence of light of a certain wavelength; or a radiation counter to detect radiation, such as a gamma counter for detection of iodine-125. For detection of an enzyme-linked secondary antibody, for example, a quantitative analysis of the amount of bound agent can be made using a spectrophotometer such as an EMAX Microplate Reader (Molecular Devices, Menlo Park, Calif.) in accordance with the manufacturer's instructions. If desired, the assays of the invention can be automated or performed robotically, and the signal from multiple samples can be detected simultaneously.

Diagnostic and prognostic techniques utilizing micro-aspirates of sample are provided. These techniques are minimally invasive and can augment conventional imaging and provide a method to greatly improve the diagnosis, staging and understanding of the pathobiology of retinal and vitreous disorders. Sensitive and precise reverse phase protein array (RPA) proteomic technology that can measure the post-translationally modified state of several hundred low abundance proteins and peptides in 20 microliters of vitreous fluid is provided. Reverse phase protein microarray is a technique that can be used for efficient and accurate detection of proteins in a sample. The proteins extracted from a single sample are immobilized on the substratum. The captured biomarkers are detected with a primary antibody directed toward the protein/polypeptide of interest and a second tagged molecule is incorporated for detection. Each spot on the array corresponds to a different sample. Total lysates of different samples are immobilized on the array and incubated with one antibody. Each spot on the array corresponds to a different sample (up to 640 lysates per array). Reverse phase microarray allows for probing into the networking and cross-talk between proteins involved in intracellular signaling. Uses of reverse phase microarray techniques in, for example, microarray printing, protein detection, and/or protein quantification are all commensurate with the scope of the present invention.

In some aspects, the detecting comprises a proteomic fingerprint comprising a vitreous fluid polypeptide or unique fragment thereof. Examples of biomarkers that have been isolated from ocular fluids are shown in Table 2 to Table 13 of U.S. Patent Application Publication 20070224644 hereby incorporated by reference in its entirety. These can be obtained by running a sample of ocular fluid on an SDS-PAGE gel, and then digesting the entire gel lane from high to low protein molecular weight with trypsin, followed by MS/MS analysis. Examples of biomarkers include retinol binding protein-4 (RBP4), Secreted Protein Acidic and Rich in Cysteine (SPARC), Akt., VEGFR, EGFR, Bcr-Abl, Her2-Neu (erbB2), TGFR, BAD, Bcl-2, C-abl, CC9, Fadd, FGF-R, PDGFR, PDGFR, VEGFA, VEGFR, C-kit, PEDF, MMP-2, MMP-9, TNF-alpha, IL-1B and eNOs.

The present invention also provides and includes a kit for the analysis of ocular fluid samples having primary antibodies selected from at least three biomarkers secondary peroxidase conjugated antibodies; and fluorescent peroxidase substrate. The present invention further provides and includes a kit for the analysis of ocular fluid samples having primary antibodies selected from at least three biomarkers of: C-abl, CC9 D330, PDGFR Y716, PDGFR Y751, VEGFR Y951, VEGFR Y996, VEGFR Y1175, PEDF, TNF-alpha, IL-1B; secondary peroxidase conjugated antibodies; and fluorescent peroxidase substrate.

### Therapeutic Agent

Once at least one abnormal biomarker is identified and a disease is diagnosed for that abnormal biomarker, a therapeutic agent can be prescribed for treatment of the diagnosed disease. The therapeutic agent can be a pharmacologic agent already known in the art. These can include both naturally occurring substances both in purified form partially purified form, or in unpurified form. Naturally occurring substances may include proteins, nucleic acids, fatty acids, steroids and other organic compounds produced in plants, animals, microorganisms or from non-living sources. In another aspect, a therapeutic agent may be a non-naturally occurring substance including proteins, nucleic acids, fatty acids, steroids and other organic compounds. A non-naturally occurring therapeutic agent may include modified natural products or substances without naturally occurring homologues. Examples of therapies can include antibodies, receptor agonists and antagonists, signaling pathway agonists and antagonists, small molecules, proteins, nucleic acids and other active agents known in the art.

A therapeutic agent can be from any class of drugs, such as anti-angiogenesis agents, cancer treatments, anti-inflammation, molecules involved in growth, anti-apoptosis agents, steroid compounds used for reduced swelling, and monoclonal antibodies and fragments thereof. In one aspect, inhibitors of TNFalpha or inhibitors molecules in the signaling pathways of TNF-R1 or TNF-R2 are therapeutic agents. In another aspect, a therapeutic agent is an inhibitor of VEGF. In particular, such therapeutic agents include bevacizumab (Avastin®), pegaptanib (Macugen®), ranibizumab (Lucentis®), Dexamethasone (Decadron®) and triamcinolone (Aristocort®). The present invention also provides for identifying a subject who would benefit from administration of a different therapeutic agent. In such an aspect, the therapeutic agent may be something other than the previously administered treatment, for example without limitation, not bevacizumab, not pegaptanib, not ranibizumab, not Dexamethasone, or not triamcinolone.

In another aspect, a therapeutic agent can be a nucleic acid or a non-nucleic acid, such as a polypeptide or a small-molecule. In a preferred embodiment, a nucleic acid can be a polynucleotide, a polynucleotide analog, a nucleotide, or a nucleotide analog. In a more preferred embodiment, a therapeutic agent can be an antisense oligonucleotide, which are nucleotide sequences complementary to a specific DNA or RNA sequence of the present invention. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotides can be deoxyribonucleotides, ribonucleotides, or a combination of both. Nucleic acid molecules can also be used.

### EXAMPLES

Vitreous samples are collected from 16 patients. Control samples are collected from surgical patients immediately prior to pars plana vitrectomy. Five control samples are collected from patients with macular hole (n=1), epiretinal membrane (n=1), or retinal detachment (n=3). Samples are collected from ten patients with wet age-related macular degeneration and one patient with idiopathic choroidal neovascularization all requiring treatment with intravitreal bevacizumab. These eleven patients undergo vitreous sampling in the office prior to intravitreal injection with intravitreal bevacizumab. The number of intravitreal injections prior to vitreous sampling range from 0 to 5. Two patients have vitreous samples taken immediately prior to intravitreal injection and then one month later prior to re-injection. All samples are obtained at least 4 weeks after prior treatment. Six patients are characterized as non-responders and five patients as responders. Two patients (patients 2 and 7) have vitreous samples taken immediately prior to intravitreal injection and then one month later prior to re-injection. Patient 2, defined as a responder, is naïve to treatment and received feeder vessel treatment and intravitreal bevacizumab. Patient 7, a non-responder, receives 2 intravitreal bevacizumab injections prior to the first vitreous sample. The last injection is done 4 weeks before the first sample was obtained.

In each case, a topical anesthetic is followed by additional anesthetic applied to the pars plana via a cotton pled-get. A sterile eyelid speculum exposes the pars plana. Betadine 5% is applied to the pars plana and formix to achieve sterility. A 1 cc syringe with a 25 gauge needle is used to obtain a small quantity (0.05 to 0.2 cc) of liquid vitreous, being careful to avoid aspiration of any subconjunctival or surface fluid while withdrawing the needle from the eye. All specimens are frozen at −20° C. for storage until subsequent analysis by reverse phase protein microarrays.

Patients are characterized as responders versus non-responders based on response to bevacizumab treatment. Response to therapy is defined as: 1) improvement in visual acuity of 10 letters or more 2) decrease in intraretinal edema and/or subretinal fluid or decrease in central 1 mm retinal thickness by 40 microns on OCT 3) decreased area of leakage on fluorescein angiography by 50% or 4) closure of retinal angiomatous proliferation (RAP) lesion.

Patients are monitored for complications including endophthalmitis, vitreous hemorrhage, retinal detachment, and any unanticipated loss of vision during the four months following vitreous aspiration.

Total protein content of the vitreous samples is measured spectrophotometricly (Bradford method). The samples are diluted in extraction buffer (T-PER (Pierce, Indianapolis, Ind.), 2-mercaptoehtanol (Sigma, St. Louis, Mo.) and 2×SDS Tris-glycine loading buffer (Invitrogen, Carlsbad, Calif.) and are denatured by heating for 8 minutes at 100° C. prior to dilution in the microtiter plate. Briefly, the lysates are printed on glass backed nitrocellulose array slides (FAST Slides Whatman, Florham Park, N.J.) using an Aushon 2470 arrayer (Aushon BioSystems, Burlington, Mass.) equipped with 350 μm pins. Each lysate is printed in a dilution curve representing neat, 1:2, 1:4, 1:8, 1:16 dilutions. The slides are stored with desiccant (Drierite, W. A. Hammond, Xenia, Ohio) at −20° C. prior to immunostaining. (Sheehan 2005)

Human Endothelial (HE) cell lysates are treated with pervanadate and are used as a model of phosphorylated VEGF receptor sensitivity and precision. Human endothelial cells express approximately 100,000 VEGF receptors/cell. Sensitivity of the arrays for VEGFR Y951 is found to be 3660 receptor molecules. To determine inter-slide precision, HE cells treated with pervanadate are printed in duplicate on 8 slides and immunostained with anti-VEGFR Y951. Dose response curves are observed between arrays (CV %: 5.0% to 17.8%, n=8). Within run variation (n=12) is found to be within 2.0% to 18.1% for the HE+pervanadate cell lysate with good linearity (R²=0.9693).

For control microarrays, cellular lysates are prepared from A431±EGF, HeLa±Pervanadate, Human Endothelial±Pervanadate (Becton Dickinson, Franklin Lakes, N.J.) and CHO-T±Insulin (Biosource/Invitrogen, Carlsbad, Calif.) are printed on each array for quality control assessments. Human Endothelial±Pervanadate cellular lysates are printed on arrays for sensitivity and precision comparisons.

Immunostaining is performed on an automated slide stainer per manufacturer's instructions (Autostainer CSA kit, Dako, Carpinteria, Calif.). Each slide is incubated with a single primary antibody at room temperature for 30 minutes. Polyclonal primary antibodies are: VEGFR Y996, VEGFR Y1175, PDGFRβ Y716, PDGFRβ Y751, and c-KIT Y703 (Cell Signaling Technology, Danvers, Mass.). A negative control slide is incubated with antibody diluent. Secondary antibody is goat anti-rabbit IgG H+L (1:5000) (Vector Labs, Burlingame, Calif.). Total protein per microarray spot is determined with a Sypro Ruby protein stain (Invitrogen/Molecular Probes, Eugene, Oreg.) per manufacturer's directions and is imaged with a CCD camera (Alpha Innotech, San Leandro, Calif.).

Each array is scanned, spot intensity is analyzed, data is normalized, and a standardized, single data value is generated for each sample on the array (Image Quant v5.2, GE Healthcare, Piscataway, N.J.). Spot intensity is integrated over a fixed area. Local area background intensity is calculated for each spot with the unprinted adjacent slide background. This resulted in a single data point for each sample, for comparison to every other spot on the array. Each sample is printed in duplicate in a miniature dilution curve. All the data is analyzed to derive a concentration value averaged between the replicates and within the linear range of the dilution curve.

Vitreous samples are stored at 4° C. for cytospin preparation. 100 microliter of vitreous fluid is spun at 1000 rpm for 3 min in a cytocentrifuge (Thermo) loaded with SuperFrost, positively charged microscope slides (Fisher). The cytospin slides are air dried and are stained with Wright-Giemsa or H&E stain per manufacturer's instructions (Fisher).

Two-sample t-test is used to compare means between two groups when data is normally distributed, otherwise Wilcoxon Rank Sum test is used. p values less than 0.05 are considered significant. Pearson correlation coefficient is performed when data had bivariate normal probability distribution (R ver2.6.1). Spearman's correlation coefficient is conducted when data was not normal (JMP ver5.0). Unsupervised clustering analysis is performed with Ward's method two-way clustering (JMP ver 5.0).

Multiple means comparison (Kruskal Wallis Test) is used to compare values between three groups. P values less than 0.05 were considered significant.

A phosphorylated form of vascular endothelial growth factor receptor (VEGFR Y1175) is detectable in the vitreous as well as other angiogenesis related receptors and proteins (PDGFRβ Y716, PDGFRβ Y751, VEGFR Y996 and c-KIT Y703). There is a statistically significant difference (p<0.0064) in VEGFR Y1175 between the responders (mean 10.70, SEM 1.07) and non-responders (mean 4.63, SEM 0.55) and between responders and controls (mean 5.24, SEM 0.0.66). There is no statistical difference between the non-responders and controls. In addition there is a statistically significant difference in VEGFR Y996 (p<0.0366) and PDGFRβ Y751 (p<0.0024) for the responder group compared to the controls and non-responders. PDGFRβ Y716 (p<0.9597) and c-KIT Y703 (p<0.0544) are not found to be significantly associated with treatment response categories.

Phosphorylated VEGF receptor can be a marker of anti-angiogenic therapy. In two eyes (patients 2 and 7), VEGFR-Y1175 is measured from vitreous samples taken just prior to and one month following intravitreal injection of bevacizumab. Patient 2, characterized as a responder, shows a 50% decrease in the area of leakage on fluorescein angiography and decrease in the retinal thickness from 348 to 307 microns on OCT. The vision improves from 20/80 to 20/60. This patient has an initial high level of VEGFR Y1175 prior to treatment similar to others in the responder group. There is a 32 percent decrease in VEGFR-Y1175 levels after treatment with bevacizumab.

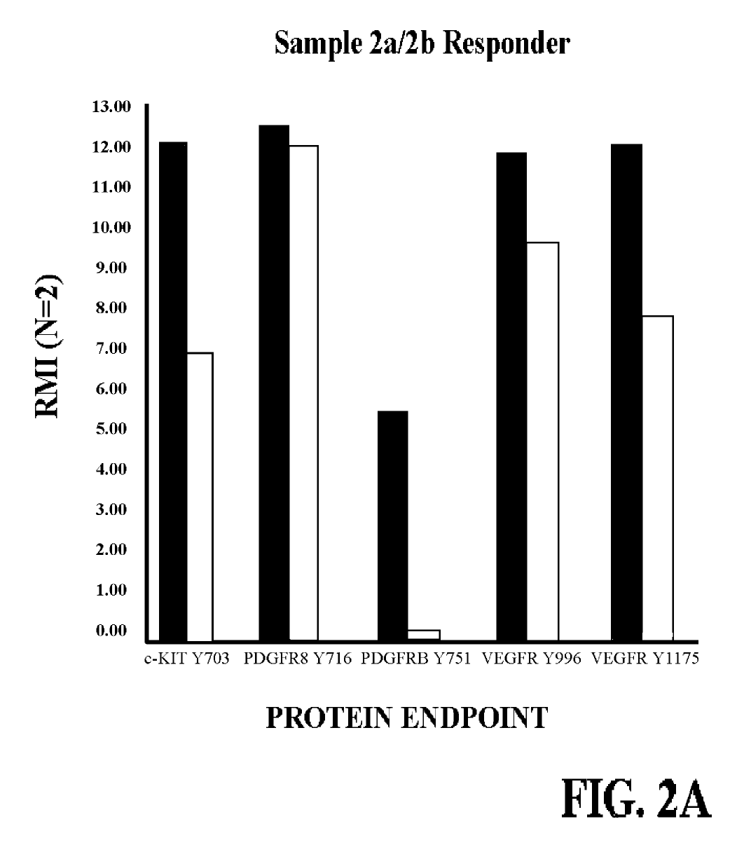

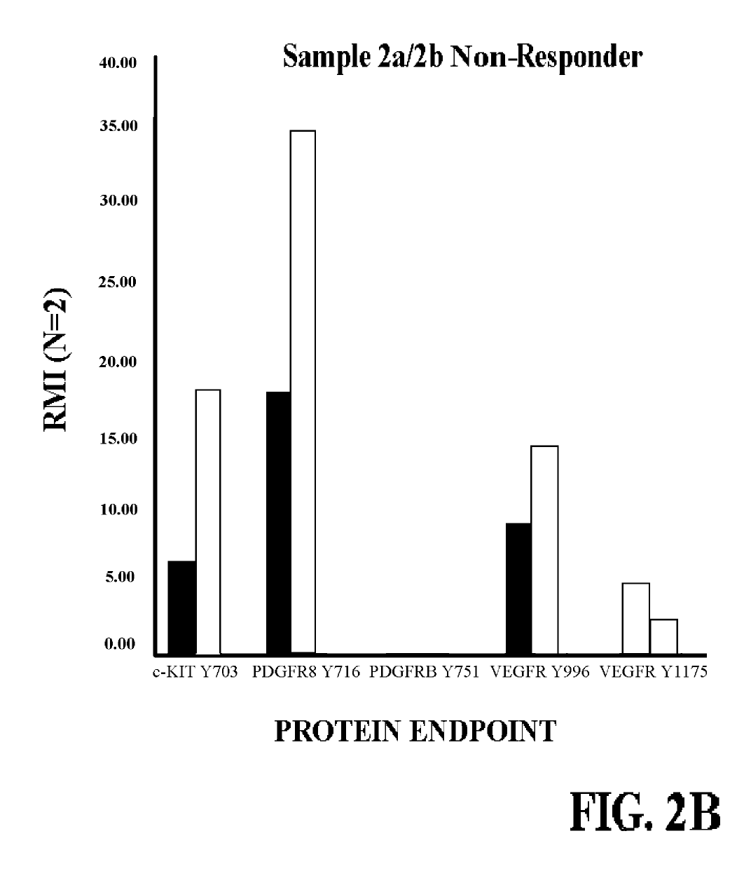

FIG. 2A and FIG. 2B shows phosphorylated angiogenesis related receptors and signal proteins in human vitreous before and after treatment with bevacizumab. In FIG. 2A, Patient 2, classified as a responder, exhibits a 32% decrease in VEGFR Y1175 one month post-bevacizumab treatment (black=pre-treatment, white=post treatment) in addition to decreases in c-KIT Y703, PDGFRβ Y716, PDGFRβ Y751, and VEGFR Y996. In FIG. 2B, Patient 7, classified as a non-responder, exhibits a 56% decrease in VEGFR Y1175 but shows concomitant increases in c-KIT Y703, PDGFRβ Y716, and VEGFR Y996 post-bevacizumab treatment. PDGFRβ Y751 levels are not above background for this patient.

Patient 7, characterized as a non-responder, shows no decrease in area of leakage on FA, increases central 1 mm retinal thickness on OCT from 361 to 422 microns, and no improvement in vision. This patient has a low level of VEGFR-Y1175 prior to treatment similar to levels found in control eyes and non-responder eyes. This patient exhibits a concomitant increase in VEGFR Y996, c-KIT Y703 and PDGFRβ Y716 post treatment. This is in direct contrast to Patient 2, who responds to treatment, and exhibits reductions in these same proteins post treatment (FIG. 2B). Although there are reductions in the target protein (VEGFR Y1175) in both of these patients, their disparate treatment response may possibly be attributed to the activity of other angiogenic related receptors that appear to increase post treatment in Patient 7.

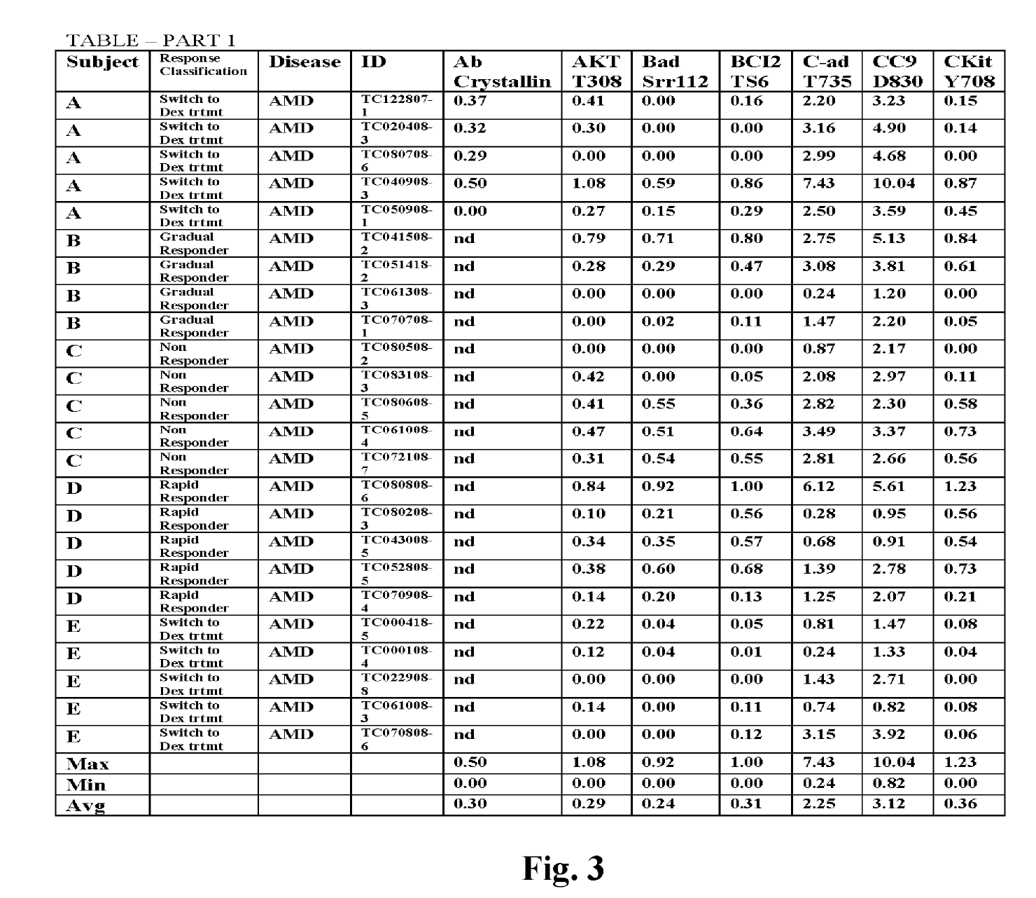

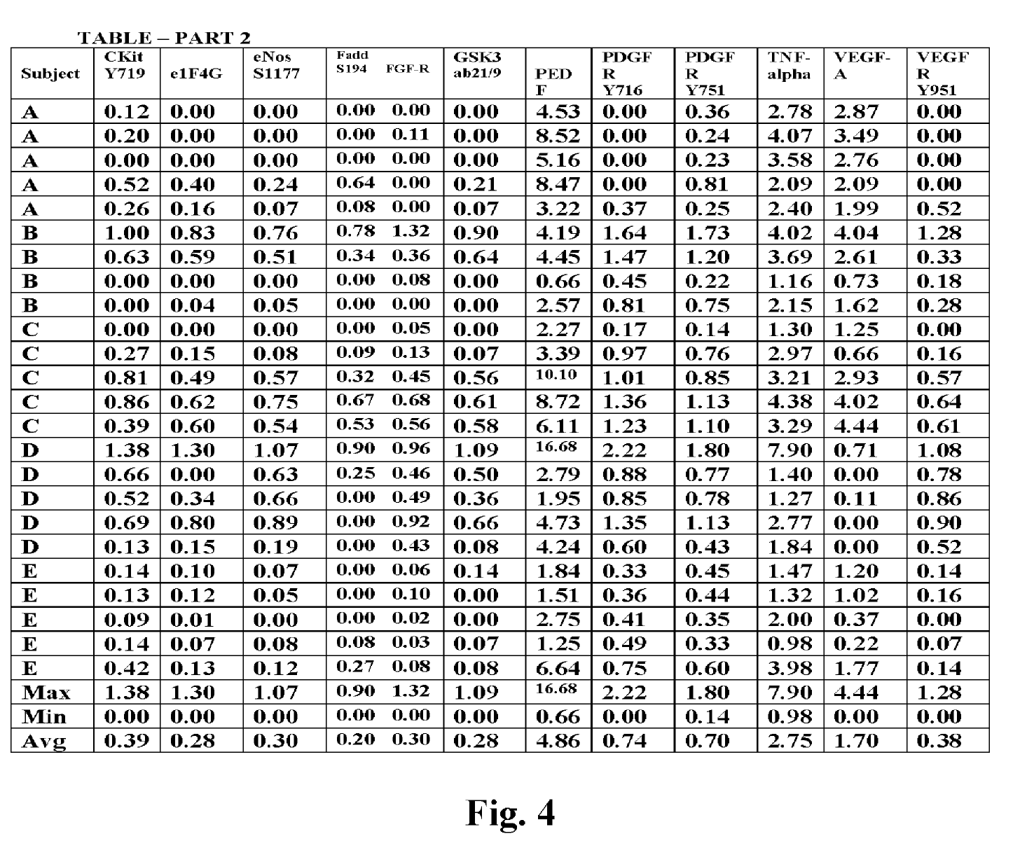

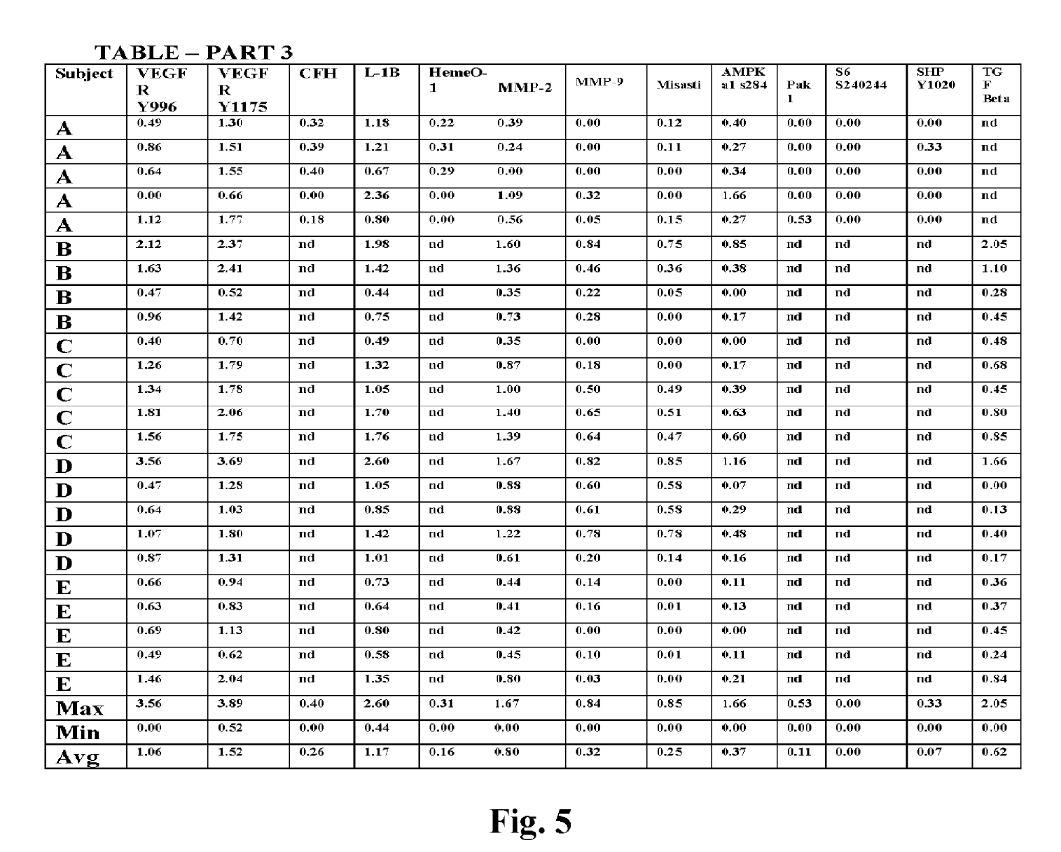

Referring to FIGS. 3, 4 and 5 in unison, three parts of a common data table are presented. The data presented shows the results of various therapeutic agents using to treat a variety of sample patients and the changes in biomarker levels created by those therapeutic agents. From the presented data, it can be shown that biomarker levels predict the rapid response of an AMD patient to bevacizumab as well as a gradual response of an AMD subject to bevacizumab. The data also shows that biomarker levels predict an RPE detachment response in an AMD patient treated with bevacizumab. In some patients, biomarker levels in an AMD subject indicate the change in therapy to Dexamethasone. The data illustrates that biomarkers can also predict the formation of a CME.

It will be understood that by drawing ocular fluid from a patent and testing that fluid, biomarkers can be found that are indicative to disease. Furthermore, once a disease is identified and being treated, the same testing protocols can be used to see if a treatment regiment is effective. In many ocular diseases, the same treatments do not work among all patients. Some patients are rapid responders, some slow responders and some non-responders to various treatments. By utilizing the present invention system and method, both the need for treatment and the effectiveness of treatment can be better controlled. It will further be understood that many aspect of the present invention system and method can be practiced in manners other than have been described. All such equivalent methods of obtaining ocular fluids and testing those fluids for biomarkers are intended to be included within the scope of the present invention as claimed.

What is claimed is:

1. A method of testing ocular fluid to provide an indication of an ocular related disease, said method comprising the steps of:
drawing an ocular fluid sample from a patient;
testing said ocular fluid sample for a level of at least one targeted biomarker, wherein said at least one targeted biomarker is selected from a group consisting of C-abl, CC9 D330, VEGFR Y1175, VEGFR Y996, PEDF, TNF-alpha, VEGFA, VEGFR Y951, IL-1B, and combinations thereof; and
comparing said level of said at least one targeted biomarker to at least one threshold level to determine if at least one abnormally elevated biomarker is present.

2. The method according to claim 1, wherein said ocular fluid is vitreous fluid.

3. The method according to claim 2, further including the step of identifying a specific ocular related disease from said at least one abnormally elevated biomarker.

4. The method according to claim 1, wherein said step of drawing an ocular fluid sample includes drawing between 5 microliters and 200 microliters of said ocular fluid from a patient.

5. The method according to claim 1, wherein said step of comparing said level of said at least one biomarker includes the substeps of:
providing access to a healthy subject;
drawing healthy ocular fluid from said subject;
testing said healthy ocular fluid to obtain a normal biomarker level; and
using said normal biomarker level as said threshold level.

6. The method according to claim 5, wherein said healthy subject is a blood relative to said patient.

7. The method according to claim 1, wherein said ocular related disease is wet Age Related Macular Degeneration.

8. The method according to claim 3, further including the step of treating said specific ocular disease identified by administering a therapeutic agent to said patient.

9. The method according to claim 8, wherein said therapeutic agent is an inhibitor of VEGF.

10. A method of screening patients for an ocular disease, comprising the steps of:
drawing vitreous fluid from at least one healthy subject;
testing said vitreous fluid to determine a normal range of a biomarker contained within said vitreous fluid, wherein said biomarker is selected from a group consisting of C-abl, CC9 D330, VEGFR Y1175, VEGFR Y996, PEDF, TNF-alpha, VEGFA, VEGFR Y951, IL-1B, and combinations thereof;
drawing a vitreous fluid sample from a patient;
testing said vitreous fluid sample for a level of at least one targeted biomarker; and
determining if said level of said at least one targeted biomarker falls within said normal range.

11. The method according to claim 10, wherein said targeted biomarker is selected from a group consisting of C-abl, CC9 D330, VEGFR Y1175, VEGFR Y996, PEDF, TNF-alpha, VEGFA, VEGFR Y951, IL-1B, and combinations thereof.

12. The method according to claim 10, further including the step of identifying an ocular disease by a targeted biomarker not being within said normal range.

13. The method according to claim 12, wherein said ocular disease is selected from a group consisting of dry age-related macular degeneration, wet age-related macular degeneration, idiopathic choroidal neovascularization, epiretinal membrane, macular hole, retinal detachment, vitreous hemorrhage, diabetic retinopathy, epiretinal membrane, proliferative diabetic retinopathy choroidal neovascularization, retinal angiomatous proliferation, glaucoma, cataract and uveitis.

14. A method of determining the effectiveness of a treatment for an ocular disease, comprising the steps of:
taking a sample of an ocular fluid from a patient;
testing said sample to determine a first level for at least one biomarker indicative of said ocular disease;
administering a therapy for said ocular disease;
taking a subsequent sample of said ocular fluid from said patient;
testing said subsequent sample to determine a second level for said at least one biomarker; and
comparing said first level to said second level to determine if levels of said at least one biomarker are changing.

15. The method according to claim 14, wherein said ocular fluid is vitreous fluid.

16. The method according to claim 14, wherein said at least one biomarker is selected from a group consisting of apoptosis factors, growth factors, angiogenesis factors and inflammation factors.

17. The method according to claim 16, wherein said apoptosis factors are selected from a group consisting of BAD Ser112, Bel-2, C-abl, CC9 D330, Fadd 5194 and combinations thereof.

18. The method according to claim 16, wherein said growth factors are selected from a group consisting of FGF-R, PDGFR Y716, PDGFR Y751, VEGFA, VEGFR, VEGFR Y951, VEGFR Y996, VEGFR Y1175 and combinations thereof.

19. The method according to claim 16, wherein said angiogenesis factors are selected from a group consisting of C-kit Y703, C-kit Y719, PEDF, MMP-2, MMP-9 and combinations thereof.

20. The method according to claim 16, wherein said inflammation factors are selected from a group consisting of TNF-alpha, IL-1B, eNOs S1177 and combinations thereof.
